(19) United States
(12) Patent Application Publication
(10) Pub. No.: US 2024/0327878 A1
(43) Pub. Date: Oct. 3, 2024

(54) PSEUDOMONAS PUTIDA STRAINS ENGINEERED FOR PRODUCTION OF MUCONIC ACID FROM P-METHOXYLATED BENZOIC ACIDS

(71) Applicant: Alliance for Sustainable Energy, LLC, Golden, CO (US)

(72) Inventors: Alissa Catherine BLEEM, Golden, CO (US); Gregg Tyler BECKHAM, Golden, CO (US); Caroline Rose AMENDOLA, Denver, CO (US)

(21) Appl. No.: 18/626,205

(22) Filed: Apr. 3, 2024

Related U.S. Application Data
(60) Provisional application No. 63/493,923, filed on Apr. 3, 2023.

(51) Int. Cl. C12P 7/18; C12N 1/20; C12R 1/38

(52) U.S. Cl. CPC C12P 7/18; C12N 1/205; C12P 2201/00; C12R 2001/38

(57) ABSTRACT

Disclosed herein are methods and compositions of matter for a homogeneous Mn and Zr autoxidation catalyst system to cleave the C—C bonds of the pine (primarily G-type lignin) and poplar (G- and S-type lignin) oligomers from RCF oils, along with the model compounds.

### CROSS-REFERENCE TO RELATED APPLICATIONS

This application claims priority under 35 U.S.C. § 119 to U.S. provisional patent application No. 63/493,923 filed on 3 Apr. 2023, the contents of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### BACKGROUND

The conversion of lignin, the heterogeneous aromatic polymer found in terrestrial plant cell walls, into renewable biochemicals is critical to enable a viable lignocellulose-based bioeconomy, which would in turn aid in mitigation of the effects of anthropogenic climate change. Extensive research for lignin depolymerization has led to numerous catalytic methods to cleave the labile aryl-ether C—O bonds in lignin, whereas cleaving recalcitrant carbon-carbon bonds has received less attention. Methods and compositions of matter disclosed herein describe a method that catalytically cleaves the C—C bonds of lignin, as shown unambiguously by using lignin oils that contain only C—C linked dimers and oligomers, to produce bio-available monomers that can be further converted to a single product through a biological funneling process.

P. putida KT2440 cannot natively utilize p-methoxylated aromatics, but these compounds are prevalent in streams of reductive catalytic fractionation (RCF) oil from lignin that have undergone methyl protection and subsequent oxidation to increase monomer yields. Integration of the catabolic pathways for p-methoxylated aromatics is essential for the realization of these streams as feedstocks for biological conversion of lignin to value-added products such as muconic acid.

### SUMMARY

In an aspect, disclosed herein is a genetically engineered Pseudomonas useful for the production of cis, cis-muconate. In an embodiment, the genetically engineered Pseudomonas of converts veratrate to cis, cis-muconate. In an embodiment, the genetically engineered Pseudomonas converts veratrate to cis, cis-muconate in a quantitative manner.

In an aspect, disclosed herein is a method for the cleavage of carbon-carbon bonds using a Mn and Zr catalyst system. In an embodiment, the Mn and Zr catalyst system comprises acetic acid as a solvent. In an embodiment, the method further comprises using oxygen as an oxidant. In an embodiment, the carbon-carbon bonds are in oligomers. In an embodiment, the oligomers are derived from pine lignin RCF oils. In an embodiment, the oligomers are derived from poplar lignin RCF oils. In an embodiment, the method further comprises a step comprising the utilization of phenol stabilization chemistry.

In an aspect, disclosed herein is a method for the cleavage of carbon-carbon bonds comprising 5-5, beta-1, beta-beta and beta-5 linkages in p-methoxylated aromatics and acetylated lignin oligomers produced from reductive catalytic fractionation using a Mn/Zr-based catalytic autoxidation system wherein the method comprises reacting the acetylated lignin oligomers produced from reductive catalytic fractionation with a mixture of a manganese salt and a zirconium salt. In an embodiment, the mixture of a manganese salt and a zirconium salt comprises Mn(OAc)₂·4H₂O and 6 mol % Zr(acetylacetonate)₄. In an embodiment, the mixture of a manganese salt and a zirconium salt are reacted with the acetylated lignin oligomers produced from reductive catalytic fractionation at a molar ratio of eight percent manganese salt and six percent zirconium salt relative to the moles of acetylated lignin oligomers. In an embodiment, the reaction of the acetylated lignin oligomers with the mixture of a manganese salt and a zirconium salt is at about 150 degrees Celsius. In an embodiment, the reaction of the acetylated lignin oligomers with the mixture of a manganese salt and a zirconium salt is under about 6 bar of oxygen pressure. In an embodiment, the reaction of the acetylated lignin oligomers with the mixture of a manganese salt and a zirconium salt reacts for up to 1.5 hours. In an embodiment, the lignin oligomers and p-methoxylated aromatics comprise vanillate, isovanillate, veratric acid and veratraldehyde. In an embodiment, the carbon-carbon bonds are labile aryl-ether bonds. In an embodiment, the Mn/Zr-based catalytic autoxidation system comprises acetic acid. In an embodiment, the Mn/Zr-based catalytic autoxidation system comprises a polar solvent. In an embodiment, the Mn/Zr-based catalytic autoxidation system comprises a non-polar solvent. In an embodiment, the method further comprises using oxygen as an oxidant. In an embodiment, the oligomers are derived from pine lignin RCF oils. In an embodiment, the oligomers are derived from poplar lignin RCF oils. In an embodiment, the method is performed in a genetically engineered bacterium. In an embodiment, the genetically engineered bacterium is Pseudomonas sp. In an embodiment, the genetically engineered bacterium is ACB263. In an embodiment, the method produces muconic acid. In an embodiment, the method produces cis, cis-muconic acid. In an embodiment, the genetically engineered bacterium is capable of metabolizing p-methoxylated aromatics.

Other objects, advantages, and novel features of the present invention will become apparent from the following detailed description of the invention when considered in conjunction with the accompanying drawings.

### BRIEF DESCRIPTION OF THE DRAWINGS

FIG. 1 depicts carbon-carbon bond linkages found in oligomers of RCF oil, depicted as dimers.

FIG. 2 depicts structures of model monomers and dimers 1-6 disclosed herein.

FIGS. 3A, 3B, 3C, 3D depict (A) GPC traces of the native pine RCF substrate (black), methylated pine RCF (dark blue), methylated pine oligomers (blue), and methylated pine distillate (light blue). (B) Comparison of the GPC traces of the pine distillate (blue) with those of 4-propylveratrole (black), 1, propanol veratrole (blue), the dimer 1,2-bis(3,4-dimethoxypehnyl)ethane (green), 2, and 2,2′,3,3′-tetramethoxy-5,5′-dipropyl-1,1′-biphenyl (orange), 5 indicating dimers present in the distillate. (C) GPC traces of the native poplar RCF substrate (black), methylated poplar RCF (dark green), methylated poplar oligomers (green), methylated poplar distillate (light green), 1-(3′,4′,5′-trimethoxyphenyl) propane, 2, and 1-(3′,4′,5′-trimethoxyphenyl) propan-3-ol. (D) Molecular structures of models of standards in panels B and C.

FIGS. 4A, 4B, 4C, 4D, 4E, 4F depict GC-FID quantification of optimizations of (A) temperature, (B) O₂ loading, (C) catalyst loading, (D) zirconium loading, (E) time, and (F) reaction concentration for the autoxidation catalysis of methylated pine RCF oligomer oxidation. Conditions except parameter changed: substrate, 50 mg; catalyst, 8 wt % Mn(OAc)₂·4H₂O, 12 wt % Zr(acac)₄; solvent, 15 mL acetic acid; O₂ loading, 6 bar; temperature, 150° C.

FIGS. 5A, 5B, 5C, 5D, 5E, 5F depict (A) GC-FID quantification of total monomers in the pine oligomers substrate (blue) and oxidation mixture (red). (B) GC-FID quantification of individual oxidation products in the pine oligomer oxidation. (C) GPC data comparing the molecular weight distribution of the pine oligomers substrate (blue) and pine oligomers oxidation (red). (D) GC-FID quantification of total monomers in the poplar oligomers substrate (blue) and oxidation mixture (red). (E) GC-FID quantification of individual oxidation products in the poplar oligomer oxidation. (F) GPC data comparing the molecular weight distribution of the poplar oligomers substrate (blue) and poplar oligomers oxidation (red). Conditions: substrate, 65 mg; catalyst, 8 wt % Mn(OAc)₂·4H₂O, 12 wt % Zr(acac)₄; solvent, 15 mL acetic acid; O₂ loading, 6 bar; time, 1.5 h; temperature, 150° C.

FIGS. 6A, 6B depict (A) a metabolic pathway for the production of muconate from oxidation streams of methylated pine RCF in P. putida strain ACB263. Aldehyde dehydrogenase(s) (ALDH) catalyze the oxidation of veratraldehyde to veratrate, which then undergoes p-demethylation to vanillate by the CYP199A4 system. VanAB O-demethylates vanillate to protocatechuate, which is decarboxylated to catechol and ring-cleaved to produce muconate. Undesirable accumulation of isovanillate was prevented by the isovanillate demethylase, IvaAB. Diversion of metabolites to growth was avoided by deletion of catRBC and pcaHG. (B) Cultivation of strain ACB263 in M9+2 mM veratraldehyde produces the equivalent molar yield of muconate. (C) Cultivation of strain ACB263 in M9+2 mM veratrate produces the equivalent molar yield of muconate. (D) Strain ACB263 produces muconate from veratrate and veratraldehyde when grown in M9+10% v/v oxidation products from methylated pine RCF (representative plot from one oxidation reaction). Catechol and isovanillate were not detected in any of the culture supernatants. Error bars indicate the standard deviation from the mean of three biological replicates.

### DETAILED DESCRIPTION

In an aspect, disclosed herein are method and compositions of matter for the treatment of lignin that has undergone HDO, autoxidation, and bioconversion.

Pseudomonas putida KT2440 strain ACB263 is derived from strain CJ781. Modifications were made to enable catabolism of the p-methoxylated aromatic compounds 3,4-dimethoxybenzoic acid and 4-methoxybenzoic acid, including integration of a cytochrome P450 P-demethylase system from Rhodopseudomonas palustris HaA2 (CYP199A4:HaPux:HaPuR) and a Rieske-type isovanillate demethylase from Comamonas testosteroni BR6020 (IvaAB). These two modifications direct the p-methoxylated compounds to vanillic acid, which is catabolized to muconic acid via a combination of native and previously engineered pathways. Strain ACB263 also utilizes a mutant of the protocatechuic acid decarboxylase, AroY(E474V), which was previously reported to enhance activity of this enzyme. Strain ACB262 contains the mutations described above, but it was built into the wild-type KT2440 background, so it grows on p-methoxylated benzoic acids as the sole source of carbon and energy instead of converting them to muconic acid.

P. putida strain CJ781 produces stoichiometric (100%) yields of muconic acid from p-coumaric acid, but it cannot utilize p-methoxylated aromatics like 4-methoxybenzoic acid or 3,4-dimethoxybenzoic acid for the production of muconic acid. Strain ACB263 retains the robust muconic acid conversion pathways while gaining the ability to catabolize p-methoxylated compounds (e.g., ACB263 produces 100% yield of muconic acid from 3,4-dimethoxybenzoic acid). P. putida strain CJ781 served as the basis for strain ACB263, it contains pathways for production of muconic acid but cannot utilize p-methoxylated compounds.

Efforts to produce aromatic monomers from catalytic lignin depolymerization have historically focused primarily on aryl-ether bond cleavage. The majority of aromatic monomers in lignin, however, are linked by various recalcitrant carbon-carbon (C—C) bonds, which present a challenging substrate for depolymerization of lignin to aromatic monomers at high yields. To increase the monomer yield in lignin depolymerization above that accessible through aryl-ether bond cleavage alone, here we report a catalytic autoxidation method to cleave C—C bonds in dimers and oligomers from lignin through aerobic autoxidation. The process specifically uses acetic acid as a solvent, a catalyst comprising manganese and zirconium salts, and oxygen as the terminal oxidant. We use phenol methylation to overcome the inhibitory effect of phenols on autoxidation. After demonstrating the process on model compounds representative of the four major types of C—C linkages in lignin, we prepared lignin substrates via reductive catalytic fractionation (RCF) of both pine and poplar, followed by distillation of the aromatic monomers from the RCF lignin oils to obtain oligomer-rich substrates with no detectable remaining aryl-ether bonds. Catalytic C—C bond cleavage reactions using the pine and poplar oligomer-rich substrates yielded 1.01 and 0.96 mmol of monomers per g of oligomer substrate, respectively, most of which are aromatic carboxylic acids. The mixtures of oxygenated monomer products from pine were converted to cis,cis-muconic acid in an engineered strain of Pseudomonas putida KT2440. Overall, autoxidation offers a catalytic strategy to increase the overall theoretical yield of valuable aromatic monomers from lignin.

Lignin is the most abundant natural source of aromatic compounds, comprising 15-30 wt % of lignocellulosic biomass, and many lignin valorization strategies to date rely on its depolymerization to aromatic monomers. Accordingly, multiple depolymerization strategies have been developed that are able to obtain theoretical yields of aromatic monomers through aryl-ether C—O bond cleavage. Compared to C—O bonds, the various C—C linkages in lignin, formed through radical processes during its biosynthesis or condensation reactions during lignocellulose processing, are more difficult to cleave.

To date, very few catalysts have been reported to cleave C—C inter-unit linkages in lignin. Notable examples of catalytic C—C bond cleavage in lignin to date include a Ru/NbOPO₄ catalyst that cleaves both C—O and C—C bonds, yielding a deoxygenated lignin oil comprising 32 wt % monomers. Others reported up to 13 wt % of phenol can be obtained through tandem C—O and C(aryl)-C bond cleavage using a multifunctional catalyst comprising CuCl₂/AlCl₃ and Ru/CeO₂. Similarly, it has been reported that an 8 wt % yield of phenol with a zeolite catalyst. Other reports of lignin C—C bond cleavage catalysis require multiple steps and demonstrate considerably lower yields.

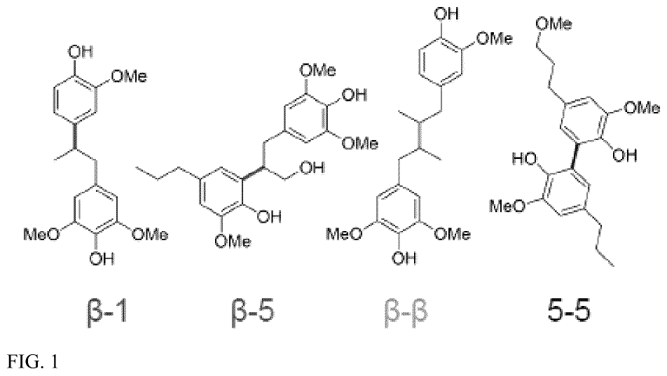

Recently, others have demonstrated that a (2,2,6,6-tetramethylpiperidin-yl)oxyl-based oxidant is able to produce 1.9 mmol of 2,6-dimethoxybenzoquinone per g of oligomers derived from reductive catalytic fractionation (RCF) of birch wood. Lignin oil from RCF is an ideal substrate to explore C—C bond cleavage in lignin due to the near-theoretical C—O bond cleavage during this process, resulting in an oil wherein the dimers and oligomers are only linked by C—C bonds, as shown in FIG. 1. Others have used 400 wt % of oxidant-to-substrate per cycle, and similar product yields can be obtained after the oxidant has been regenerated.

Despite these promising initial reports, additional systems are needed that can catalytically cleave C—C bonds in lignin to utilize lignin more effectively and offer product flexibility for downstream lignin valorization to useful products. To this end, autoxidation, the radical-chain propagated reaction involving oxygen, occurs throughout nature and has enormous industrial relevance (e.g., production of cumene, cyclohexanone and cyclohexanol, terephthalic acid and other aromatic carboxylic acids, and alkyd paints). Various metals (often Co and Mn) catalyze autoxidation by breaking down hydroperoxides via the Haber-Weiss reaction. Furthermore, the free radicals formed can undergo C—C bond cleavage via B-scission. Others have showed that the Co/Mn/Zr/Br system can cleave C—C bonds in several common plastics, and others demonstrated production of monomers from various lignin sources and aryl-ether model compounds. In companion studies to this work, we demonstrated C—C bond cleavage of both mixed plastics and an acetyl-protected poplar RCF oligomer substrate using Co and Mn autoxidation catalysis.

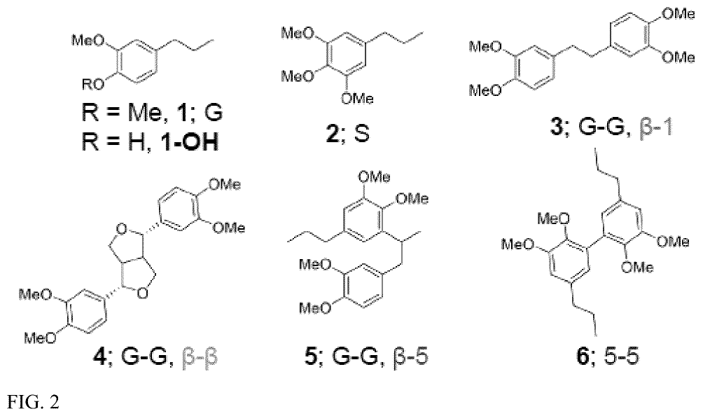

As disclosed herein, we use a homogeneous Mn and Zr autoxidation catalyst system to cleave the C—C bonds of the pine (primarily G-type lignin) and poplar (G- and S-type lignin) oligomers from RCF oils, along with the model compounds 1-6 shown in FIG. 2. Relative to our companion study using Co and Mn with acetyl-protected lignin oligomers, here we sought to understand if the cheaper and more abundant Mn would suffice for this reaction relative to Co, and we also targeted bromide-free conditions, as it is corrosive and requires specialized titanium-clad reactors. We showed that the Mn and Zr catalyst system is sufficient to activate the electron-rich lignin substrate in the absence of bromide and produce a mixture of bio-available monomers that can be converted to single products using biological funneling.

Methyl protection of phenolic groups enables lignin autoxidation and requires Zr for C—C bond cleavage. We first explored oxidations of mono-aromatic model compounds with propyl alkyl side chains (an ethylene linker in the case of 3) to test whether C—C bond cleavage can be achieved using Mn-catalyzed autoxidation. The “standard conditions” shown in Scheme 1 are those determined to be optimum for the RCF oligomer substrate (vide infra), and the yields for model compound experiments are reported in mol % aromatic compounds. The oxidation mixtures were derivatized by methylation prior to GC-FID analysis for accurate quantification of the carboxylic acids. The model compounds and analytical standards were synthesized as detailed in the ESI.

We initially attempted to oxidize 4-propylguaicol, 1-OH, a coniferyl (G)-type monomer, as shown in Scheme 1. Treatment of 1-OH at 6 bar O₂ and 150° C. with 8 mol % Mn(OAc)₂·4H₂O and 6 mol % Zr(acac) (acac=acetylacetonate) as catalysts in a steel reaction vessel resulted in 94 (1) % conversion of 1-OH to several products, but only 2.1 (6) mol % of vanillin was obtained as a result of C—C bond cleavage. The product of C—C bond coupling, divanillin, was produced in 11(3) mol % yield along with 4.0 (5) mol % of 4-propyl-2-methoxy-6-hydroxyphenyl acetate, apparently due to ring hydroxylation.

Due to the low recovery of aromatic products with 1-OH, we next sought oxidations of the methyl-protected analogue of 4-propylguaiacol, 1-OH, namely, 4-propylveratrole, 1, along with 1-(3′,4′,5′-trimethoxyphenyl) propane, 2, the methyl-protected analogue of the syringyl(S)-type 4-propylsyringol, as shown in Scheme 1. Treatment to 1 at the standard oxidation conditions yielded 30 (8) mol % of products comprising 13 (7) mol % of veratric acid, 5.5 (3) mol % of veratraldehyde, 5.1 (9) mol % of 2-acetoxypropioveratrone, and 6.2 (5) mol % of 2-methoxymaleic acid, the lattermost evidently arising from ring opening of 1. Similarly, 2 exhibited a total product yield of 48 (2) mol % aromatic products, including a 14.5 (6) mol % yield of 3,4,5-trimethoxybenzaldehyde and 23 (1) mol % yield of 3,4,5-trimethoxybenzoic acid. Like the reaction of 1, the 2-acetoxy aryl ketone was also produced, in 10.5 (5) mol % yield. In the absence of the Zr co-catalyst, oxidation of 1 yielded only trace quantities of the partially oxidized erythro- and threo 1-(3,4-dimethoxyphenyl)-1,2-diacetoxypropanes.

Scheme 1. Autoxidation of monomer models 1-OH, 1, and 2, with yields shown as value (standard deviation) in mol % aromatics. Conditions: substrate, 0.1 mmol; catalyst, 8 mol % Mn(OAc)2·4H2O, 6 mol % Zr(acac)₄; solvent, 15 mL acetic acid; O2 loading, 6 bar; time, 1.5 h; temperature, 150° C.

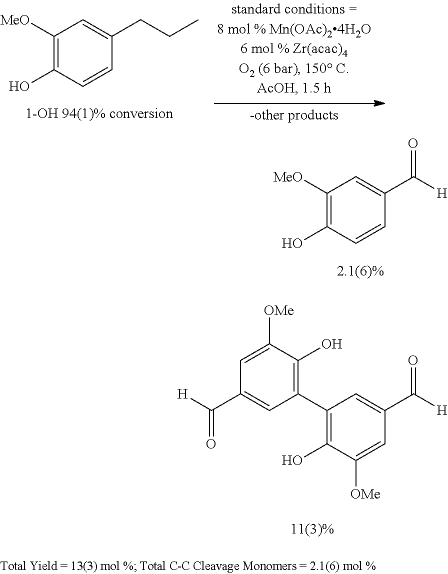 [SMILES: CCCc1ccc(O)c(OC)c1 ; COc1cc(C=O)ccc1O ; COc1cc(C=O)cc(-c2cc(C=O)cc(OC)c2O)c1O]

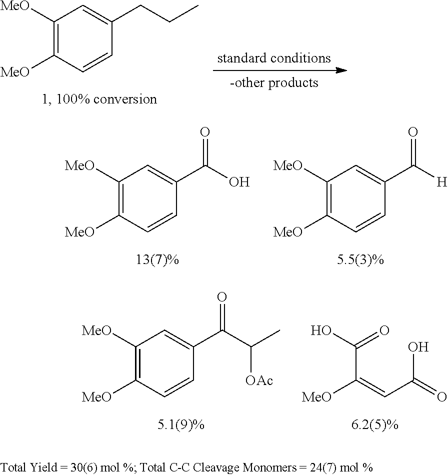 [SMILES: CCCc1ccc(OC)c(OC)c1 ; COc1ccc(C(=O)O)cc1OC ; COc1ccc(C=O)cc1OC ; COc1ccc(C(=O)C(C)OC(C)=O)cc1OC ; CO/C(=C/C(=O)O)C(=O)O]

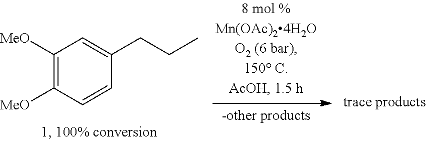

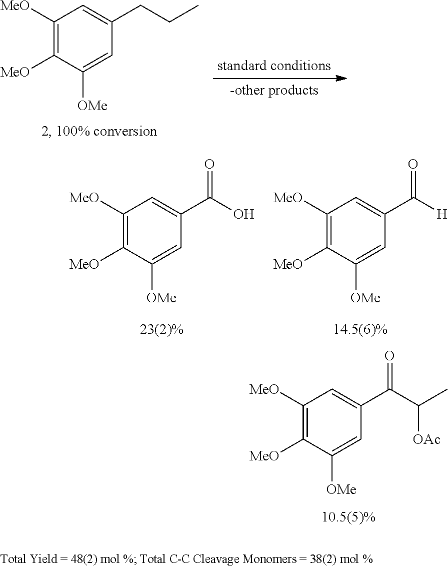 [SMILES: CCCc1cc(OC)c(OC)c(OC)c1 ; COc1cc(C(=O)O)cc(OC)c1OC ; COc1cc(C=O)cc(OC)c1OC ; COc1cc(C(=O)C(C)OC(C)=O)cc(OC)c1OC]

The aromatic acid and aldehyde products exhibit low stability under the catalytic conditions. Subjecting the S- and G-type aromatic aldehyde and aromatic carboxylic acid oxidation products to the catalysis conditions demonstrated low aromatic product recovery, as shown in Scheme 2. For example, veratric acid was recovered in 62(7) mol %, whereas veratraldehyde was recovered in 38 mol % (5(1) mol % veratraldehyde and 33(4) mol % as veratric acid). Similarly, 3,4,5-trimethoxybenzoic acid was recovered in 51(12) mol %, and 3,4,5-trimethoxybenzaldehyde was recovered in 43 mol % (20 mol % aldehyde and 23 mol % as 3,4,5-trimethoxybenzoic acid). Each substrate produced 2-methoxymaleic acid, which is indicative of oxidative ring-opening.

Scheme 2. Product stability reactions under autoxidation conditions with yields shown as value (standard deviation) in mol % aromatics. Conditions: substrate, 0.1 mmol; catalyst, 8 mol % Mn(OAc)₂·4H₂O, 6 mol % Zr(acac)₄; solvent, 15 mL acetic acid; O₂ loading, 6 bar; time, 1.5 h; temperature, 150° C.

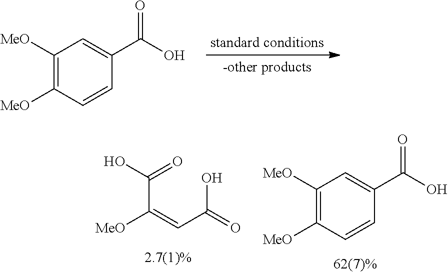 [SMILES: COc1ccc(C(=O)O)cc1OC ; CO/C(=C/C(=O)O)C(=O)O ; COc1ccc(C(=O)O)cc1OC]

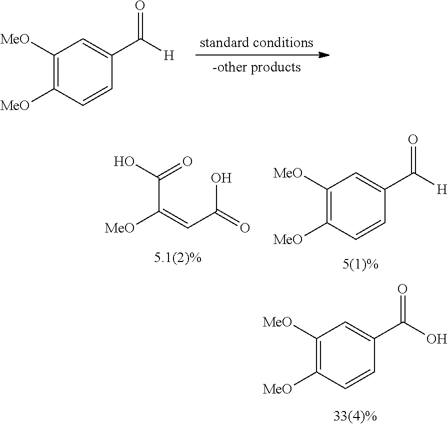 [SMILES: COc1ccc(C=O)cc1OC ; CO/C(=C/C(=O)O)C(=O)O ; COc1ccc(C=O)cc1OC ; COc1ccc(C(=O)O)cc1OC]

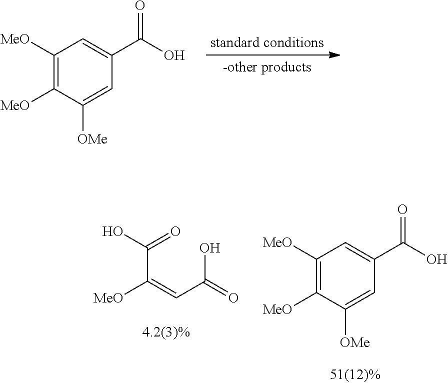 [SMILES: COc1cc(C(=O)O)cc(OC)c1OC ; CO/C(=C/C(=O)O)C(=O)O ; COc1cc(C(=O)O)cc(OC)c1OC]

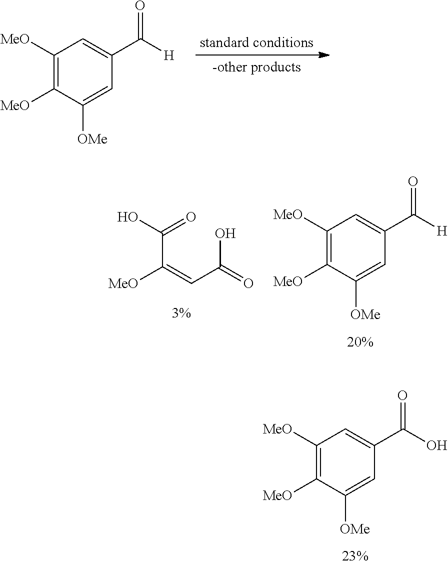 [SMILES: COc1cc(C=O)cc(OC)c1OC ; CO/C(=C/C(=O)O)C(=O)O ; COc1cc(C=O)cc(OC)c1OC ; COc1cc(C(=O)O)cc(OC)c1OC]

Dimer model compound oxidation reactions with the Mn/Zr catalyst system demonstrate monomer production through C—C bond cleavage. In a similar manner, the methylated dimers 3-6 (FIG. 2) were oxidized using the standard reaction conditions, as shown in Scheme 3. Fortunately, we discovered this catalyst system can liberate monomers from three of the four types of C—C bonds studied (β-1, β-5, β-β).

The model β-1 dimer, 1,2-bis (3,4-dimethoxyphenyl)ethane, 3, when oxidized, gave a total product yield of 35(4) mol % aromatics consisting of 29(3) mol % of veratric acid, 2.0 (2) mol % veratraldehyde, and 4.4(4) mol % of 2-methoxymaleic acid. Oxidation of the β-β model compound eudesmin, 4, afforded a total product yield of 45(2) mol % comprising 37(2) mol % veratric acid, 4.3(6) mol % veratraldehyde, and 2.9(2) mol % methoxymaleic acid. The β-5 dimer, 5, gave a total product yield of 30(1) mol % of veratric acid (12.5(8) mol %), veratraldehyde (15(1) mol %), 2-methoxymalelic acid (2.0(4) mol %), along with trace quantities of 3,4-dimethoxyisophthalic acid.

The 5-5 dimer model, 1,1′-dipropyl-3,3′,4,4′-tetramethoxybiphenyl, 6, did not yield monomer products in significant quantities and afforded 30(1) mol % of products comprising 13.7(7) mol % of the dimethyl divanillate, 7.6(4) mol % of dimethyl divanillin, and 6.9(4) mol % of the mixed acid/aldehyde analogue. Additionally, 2.1(1) mol % of the monocyclic 3,4-dimethoxyisophthalic acid was identified evidently due to ring cleavage of one of the aromatic units.

Scheme 3. Autoxidation of dimer models 3-6 with yields shown as value (standard deviation) in mol % aromatics. Conditions: substrate, 0.1 mmol; catalyst, 8 mol % Mn(OAc)₂·4H₂O, 6 mol % Zr(acac)₄; solvent, 15 mL acetic acid; O₂ loading, 6 bar; time, 1.5 h; temperature, 150° C.

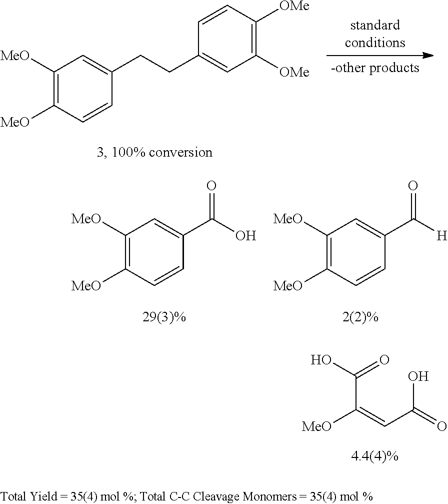 [SMILES: COc1ccc(CCc2ccc(OC)c(OC)c2)cc1OC ; COc1ccc(C(=O)O)cc1OC ; COc1ccc(C=O)cc1OC ; CO/C(=C/C(=O)O)C(=O)O]

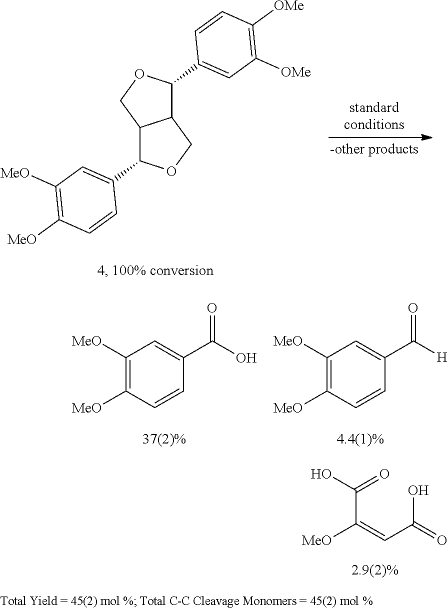 [SMILES: COc1ccc([C@H]2OCC3C2CO[C@@H]3c2ccc(OC)c(OC)c2)cc1OC ; COc1ccc(C(=O)O)cc1OC ; COc1ccc(C=O)cc1OC ; CO/C(=C/C(=O)O)C(=O)O]

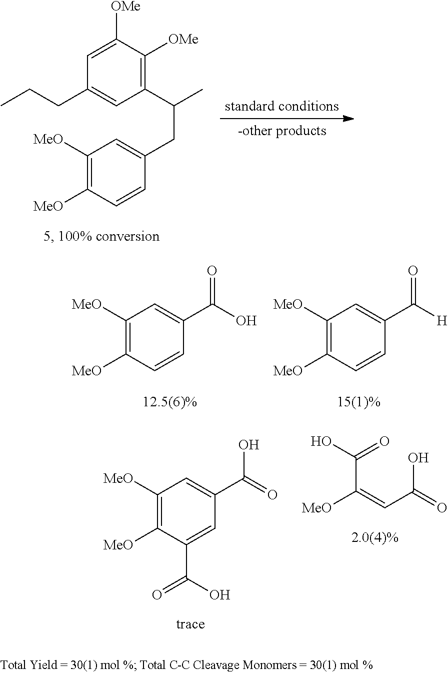 [SMILES: CCCc1cc(OC)c(OC)c(C(C)Cc2ccc(OC)c(OC)c2)c1 ; COc1ccc(C(=O)O)cc1OC ; COc1ccc(C=O)cc1OC ; COc1cc(C(=O)O)cc(C(=O)O)c1OC ; CO/C(=C/C(=O)O)C(=O)O]

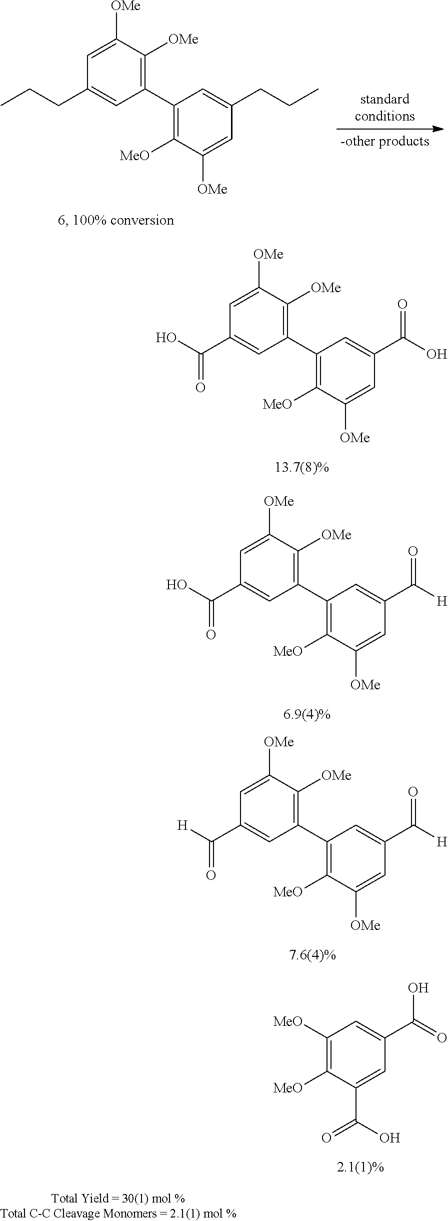 [SMILES: CCCc1cc(OC)c(OC)c(-c2cc(CCC)cc(OC)c2OC)c1 ; COc1cc(C(=O)O)cc(-c2cc(C(=O)O)cc(OC)c2OC)c1OC ; COc1cc(C=O)cc(-c2cc(C(=O)O)cc(OC)c2OC)c1OC ; COc1cc(C=O)cc(-c2cc(C=O)cc(OC)c2OC)c1OC ; COc1cc(C(=O)O)cc(C(=O)O)c1OC]

Lignin from distillation of poplar and pine RCF oils are a monomer-free substrate for autoxidation experiments. RCF was used to produce lignin oils from pine and poplar using a 5 wt % Ru/C catalyst in methanol at 225° C. for 6 h with 30 bar H2. The total yields of monomers were quantified by GC-FID to be 1.5 mmol/g of phenolic pine RCF oil, and 1.8 mmol/g of poplar oil. Phenol protection by methylation of the RCF oils was accomplished by stirring the oils in acetonitrile with excess potassium carbonate and methyl iodide at room temperature for 60 h. For pine, the monomers were recovered quantitatively as their methylated analogues. For poplar, near quantitative recovery was observed for all monomers except the S-type monomers 4-propenyl-2,6-dimethoxyphenol (44% recovery) and 3-(4-hydroxy-3,5-dimethoxyphenyl)-1-propanol (8% recovery). Residual phenols in the methylated oils were not detected ³¹P NMR.

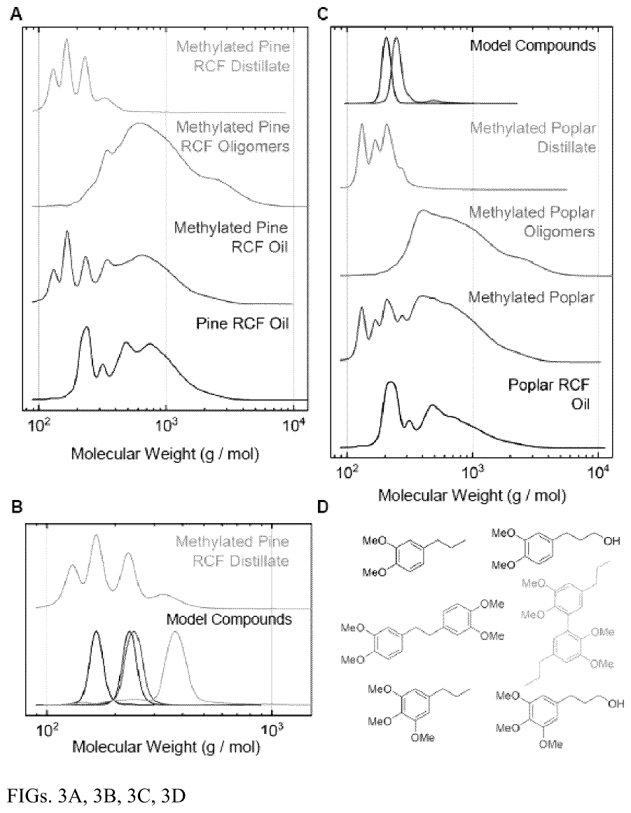

Distillations of the methylated oils were performed at 220° C. and 10 mbar for 30 min. The GC-FID quantification data of monomers in the oligomer and distillate fractions from the pine distillation showed a monomer recovery of 98% in the distillate collection flask with <1% remaining in the bottoms. The distillation of poplar also gave high recoveries: 95% of the monomers were recovered with only 3% detected in the bottoms fraction. The gel permeation chromatography (GPC) data are consistent with the quantification data and the successful removal of monomers to produce oligomer-enriched substrates for both pine and poplar, as shown in FIG. 3. A combination of GPC, GC-FID, and GC-MS data on the pine distillate and analytical standards shown in FIG. 3B indicated that dimers are present in the distillate.

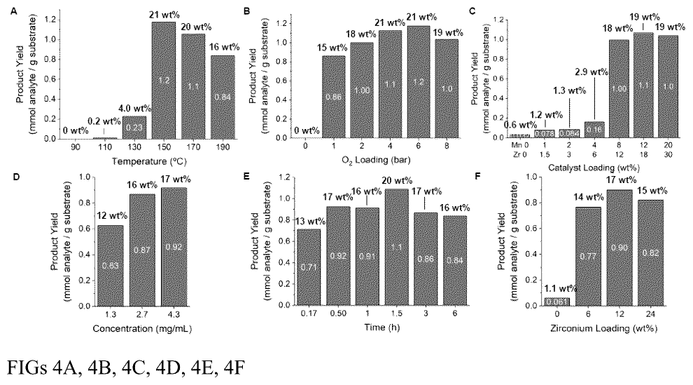

Optimal reaction conditions to maximize monomer yields were determined using the methylated pine RCF oligomer substrate. Using the methylated oligomer substrates, we next sought to optimize the autoxidation catalysis conditions, including reaction temperature, oxygen loading, catalyst loading, substrate loading, reaction time, and Zr loading, as shown in FIG. 4. As done for the model compounds, the oxidation mixtures containing carboxylic products were derivatized by methylation prior to GC-FID analysis (ESI Methods). The yields are reported in mmol analyte/g of substrate in the bar charts with wt % (g analytes/g substrate×100) yields reported above each bar. A screen of six reaction temperatures (FIG. 4A) ranging 90-190° C. showed a distribution of yields that increased sharply from 0.23 mmol/g of oligomer substrate at 130° C. to 1.2 mmol/g at 150° C., at which temperature the maximum yield was observed. At 170° C., the yield decreased to 1.1 mmol/g and to 0.84 mmol/g at 190° C. The oxygen partial pressure also affected monomer yields. At PO₂=0 bar, no monomers were detected, as expected. There was an increase in total monomer yield from PO₂=1 bar, which gave a yield of 0.89 mmol/g of oligomer substrate, to 1.2 mmol/g at 6 bar. An oxygen loading of PO2=8 bar did not improve the overall yield. An increase in total monomer yield was also observed with increased substrate concentrations (in 15 mL acetic acid) and catalyst loadings. While maintaining a constant catalyst loading of 8 wt % Mn(OAc)₂·4H₂O and 12 wt % Zr(acac)₄, an increase in the total monomer yield was observed from 0.63 mmol/g with a 3.1 mg/mL concentration to 0.92 mmol/g with a 4.3 mg/mL concentration. Similarly, when changing the catalyst loading while maintaining a substrate quantity of 50 mg, the total product yields increased from 0.032 mmol/g of pine oligomer substrate with 0 wt % catalyst to 0.16 mmol/g with 4 wt % Mn(OAc)₂·4H₂O and 6 wt % Zr(acac)₄. Using 8 wt % Mn(OAc)₂·4H₂O and 12 wt % Zr(acac)₄, a significant increase in product was observed (1.00 mmol/g of oligomer substrate). Higher catalyst loadings did not produce significantly higher product yields.

A time course study demonstrated an increase in the total monomer yield from 0.71 mmol/g at 10 min to 0.92 mmol/g at 30 min (FIG. 4E). Between 30 min and 1.5 h, similar yields ranging 0.91-1.1 mmol/g were observed. Longer reaction times do not appear to increase the total monomer yield. Lastly, we studied the effect of Zr loading. As shown with 1 in model compound studies in Scheme 1, the presence of Zr(acac)₄ significantly impacted the yield of products (FIG. 4F). Similarly, when Zr(acac)₄ was omitted from the oligomer oxidation reaction, only 0.061 mmol/g of identifiable products were quantified (primarily veratric acid and aldehyde). With a loading of 6 wt % Zr(acac)₄, the overall yield increased to 0.77 mmol/g, and 12 wt % yielded 0.90 mmol/g. No significant increase was observed with 24 wt % Zr(acac)₄.

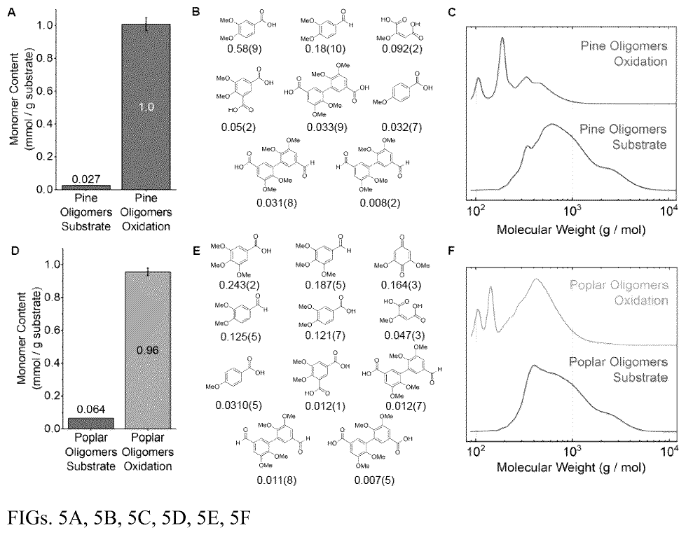

Using the optimized conditions identified from the experiments presented in FIG. 4, a total monomer yield of 1.0(4) mmol/g of oligomer substrate was obtained for the oxidation of the methylated pine oligomer substrate, as shown in FIG. 5A-C. For poplar, we applied the same conditions and obtained 0.96(2) mmol/g, as shown in FIG. 5D-F. The 1.01(4) and 0.96(2) mmol/g total monomer yields obtained from pine and poplar, respectively, represent 28 mol % of the total monomer yield of the respective RCF processes. A representative calculation for pine is presented in eq 1 and 2 below.

$$\frac{1.01 mmol monomer}{g pine oligomer substrate} \times \frac{0.4 g pine oligomer substrate}{g pine methylated RCF} \times \frac{1.065 g pine methylated RCF}{g pine phenolic RCF} = \frac{0.43 mmol monomer}{g pine phenolic RCF} \qquad ( 1 )$$

$$( \frac{0.43 mmol monomer}{g pine phenolic RCF} ) / ( \frac{1.52 mmol monomer}{g pine phenolic RCF} ) \times 100 = 28 \% \qquad ( 2 )$$

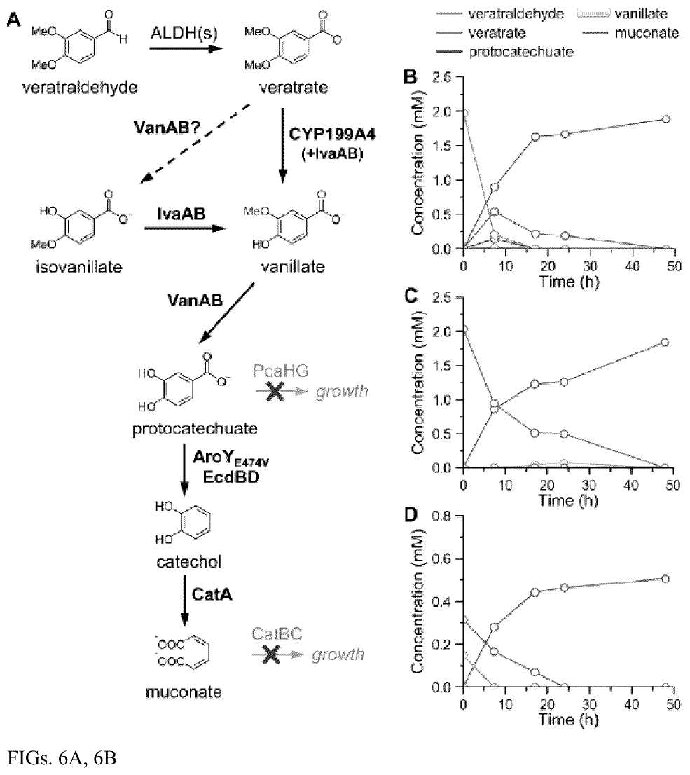

Biological conversion enables the conversion of the pine oxidation mixture to cis, cis-muconate. Aromatic-catabolic microbes can catabolize heterogenous mixtures of aromatic compounds toward a single target product through a process termed biological funneling. The oxidative catalytic approach described above generates a slate of low molecular weight, water-soluble products, which are ideal for use as microbial growth substrates. Here, strains of Pseudomonas putida KT2440 were engineered to produce cis,cis-muconic acid from the aromatic monomers in the oxidation streams of methylated pine oligomers. As shown in FIG. 5A, veratric acid and veratraldehyde comprised a large fraction (0.76 mmol/g substrate) of these streams, but these p-methoxylated compounds are not natively assimilated by P. putida. Fortunately, a cytochrome P450 monooxygenase from Rhodopseudomonas palustris HaA2 catalyzes p-demethylation of veratrate, so this operon (CYP199A4-HaPux-HaPuR) was genomically integrated into P. putida under control of the strong Ptac promoter with optimized ribosome binding sites for each gene, generating strain ACB236 which has a genotype of P. putida KT2440 ΔPP_5322::P_{tac}:CYP199A4-HaPux_{newRBS}-HaPuR. Additionally, preliminary experiments indicated that the native vanillate demethylase, VanAB, could O-demethylate veratrate to isovanillate (FIG. 6A), so the isovanillate demethylase operon ivaAB48 was integrated to prevent undesirable accumulation of isovanillate, resulting in P. putida strain ACB262.

To ensure that P. putida could utilize the major constituents of the oxidized RCF oligomer stream, strain ACB262 was cultivated in M9 minimal medium with 2 mM veratrate or 2 mM veratraldehyde as the sole source of carbon and energy. In both cases, the model compound was fully consumed within 50 h, and no accumulation of isovanillate was observed. Next, strain ACB262 was evaluated for its ability to grow with oxidized oligomer streams as the sole sources of carbon and energy. Methylated oligomers of pine RCF oil were oxidized in triplicate at the 65 mg scale, treated with aqueous base to precipitate catalyst, neutralized, and filter sterilized (ESI Methods). The resulting solutions were added at 10% v/v to M9 minimal medium. When P. putida ACB262 was grown in M9 with 10% v/v of oxidized RCF oligomers, it simultaneously utilized both veratrate and veratraldehyde from these streams without the need for a supplemental carbon source and without accumulation of intermediates.

Additional metabolic engineering was required to produce muconate from veratrate and veratraldehyde in P. putida. A previously described strain, CJ781,46 was modified to include the optimized CYP199A4-HaPux-HaPuR and ivaAB operons for p-demethylation as described above, as well as the mutant AroYE474V for decarboxylation of protocatechuate to catechol (FIG. 6A). 49 This resulted in the non-naturally occurring P. putida strain ACB263. To verify functionality of the engineered pathway, strain ACB263 was grown with the model compounds veratrate and veratraldehyde, and glucose was added to 5 mM every 24 h to support growth. Each aromatic substrate was consumed within 48 h and fully converted to muconate at 100% yield with minimal accumulation of intermediates (FIG. 6B-C). Strain ACB263 was also cultivated with the same oxidized RCF oligomer streams as ACB262, and it fully converted the major monomer constituents (veratrate, veratraldehyde) to muconate within 24 h, again achieving 100% of the theoretical muconate yield from these two substrates (FIG. 6D). Uninoculated control media incubated under the same conditions did not exhibit considerable changes in composition, indicating that all measured changes in analytes were associated with the action of the bacterium. Of note, p-methoxybenzoate was detected in small amounts in the oxidation streams of methylated pine RCF (FIG. 5A), and it is a viable substrate for the CYP199A4 system. However, p-methoxybenzoate was not analyzed because its contribution to the total muconate yield was assumed to be negligible in these dilute biological preparations.

These results demonstrate the catalytic production of bio-available monomers through C—C bond cleavage, as shown unambiguously using oligomers prepared from pine and poplar RCF oils, and their biological conversion to a single product, cis,cis-muconic acid. This autoxidation catalyst system allows cleavage of the aryl propane units in 3 of the 4 types of carbon-carbon linkages present in lignin, shown in FIG. 1 (β-1, β-5, β-β). While this is promising as the reagents used are inexpensive and the conditions are readily accessible, the overall process performed in batch reactors is limited by the need for stabilization chemistry and low product stability, as observed in our model compound oxidations in Schemes 1-3. Although autoxidation catalysis cannot be performed on lignin-derived phenolic compounds in the absence of phenol protection, there are several options that could facilitate progressing this chemistry further. We note that while the following discussion mostly pertains to the tandem RCF-autoxidation chemistry described in this work, the autoxidation catalysis approach could likely be applied to lignin substrates prepared by other processes, which will be examined in future work.

One approach to improve the viability of lignin methylation could be to use a flow system with dimethyl carbonate as a methylating reagent. Further, the regeneration of dimethyl carbonate from carbon dioxide is an active area of study and would enable a circular methylation process. Additionally, the overall delignification of biomass in the RCF process can be improved using by water as a cosolvent with an alcohol, which could elevate delignification percentages as high as 95% and would improve the overall mass yield from biomass of this process.

To overcome the issue of aromatic decomposition, the products could be continuously removed from the reaction in a flow reactor as they are generated, which could allow us to perform lignin oxidation with monomers present. Alternatively, if the propyl chain RCF monomers are sufficiently valuable, other separation methods could be employed including advanced chromatographic techniques and membrane separations that allow the retention of RCF dimers in the oligomer substrate.

Although our system does not produce appreciable monomers from 5-5 dimers, a metabolic pathway for cleavage of the 5-5 C—C bond has been reported. The resulting monomers—equivalents of vanillate and 4-carboxy-2-hydroxypenta-2,4-dienoate (CHPD), in the case of 5,5′-dehydrodivanillate cleavage—could then be directed toward central metabolism or engineered pathways for muconate production in P. putida.

The role of Zr in the oxidation of the RCF oligomers is not obvious based on product yields and distributions. In the MC-process, Zr is used as a promoter, especially for the oxidation of poly(alkyl)benzenes. Several hypotheses based on kinetics data involving Co have been proposed to explain the increase in activity when Zr is added. While it is unclear whether these models can be extrapolated to our Mn catalyst and lignin-derived substrate, we observed an increase in selectivity toward reactions that produce the desired acid and aldehyde products as opposed to deleterious side reactions.

Oxidation specifically allows the production of bio-available compounds that can be funneled into a single product, in this case, cis, cis-muconic acid, a platform chemical used in the production of biopolymers including nylon, nylon derivatives, and polyethylene terephthalate (PET), as well as performance-advantaged composites. Translation of the CYP199A4 system from R. palustris HaA2 provides a catabolic pathway for p-methoxylated aromatics in engineered P. putida, and the use of oxidized RCF oligomers offers an example of biological funneling of a heterogenous, lignin-derived stream toward a single product. The oxidized RCF oligomer streams were provided to P. putida at dilute concentrations, but the strains rapidly utilized the major monomer constituents (veratrate and veratraldehyde), indicating that tolerance to and conversion of higher substrate loadings is within the performance capability of these strains, as we have demonstrated for similar feedstocks and engineered pathways. Methylated RCF distillates (pre-oxidation) could also be provided to P. putida as a source of bio-available monoaromatics, increasing the yield of muconate or similar products from lignin. Going forward, experimental design considerations could be combined with metabolic engineering or evolution strategies to further improve the performance of these biocatalysts.

As disclosed herein, we have demonstrated unambiguous C—C bond cleavage using a Mn and Zr catalyst system in acetic acid, as shown with oligomers derived from pine and poplar lignin RCF oils. The method uses molecular oxygen as the oxidant and phenol stabilization chemistry and yields of 1.01 (4) and 0.96 (2) mmol of monomers per g of oligomer substrate were obtained for pine and poplar, respectively. The oxygenated and bioavailable product stream was converted in biological systems using P. putida KT2440, as demonstrated by the quantitative conversion of veratrate in the pine oxidation streams to cis,cis-muconate.

Additional embodiments are disclosed in Appendix A attached hereto.

The foregoing disclosure has been set forth merely to illustrate the invention and is not intended to be limiting.

We claim:

1. A method for the cleavage of carbon-carbon bonds comprising 5-5, beta-1, beta-beta and beta-5 linkages in p-methoxylated aromatics and acetylated lignin oligomers produced from reductive catalytic fractionation using a Mn/Zr-based catalytic autoxidation system wherein the method comprises reacting the acetylated lignin oligomers produced from reductive catalytic fractionation with a mixture of a manganese salt and a zirconium salt.

2. The method of claim 1 wherein the mixture of a manganese salt and a zirconium salt comprises Mn(OAc)₂·4H₂O and 6 mol % Zr(acetylacetonate)₄.

3. The method of claim 2 wherein the mixture of a manganese salt and a zirconium salt are reacted with the acetylated lignin oligomers produced from reductive catalytic fractionation at a molar ratio of eight percent manganese salt and six percent zirconium salt relative to the moles of acetylated lignin oligomers.

4. The method of claim 1 wherein the reaction of the acetylated lignin oligomers with the mixture of a manganese salt and a zirconium salt is at about 150 degrees Celsius.

5. The method of claim 1 wherein the reaction of the acetylated lignin oligomers with the mixture of a manganese salt and a zirconium salt is under about 6 bar of oxygen pressure.

6. The method of claim 1 wherein the reaction of the acetylated lignin oligomers with the mixture of a manganese salt and a zirconium salt reacts for up to 1.5 hours.

7. The method of claim 1 wherein the lignin oligomers and p-methoxylated aromatics comprise vanillate, isovanillate, veratric acid and veratraldehyde.

8. The method of claim 1 wherein the carbon-carbon bonds are labile aryl-ether bonds.

9. The method of claim 1 wherein the Mn/Zr-based catalytic autoxidation system comprises acetic acid.

10. The method of claim 1 wherein the Mn/Zr-based catalytic autoxidation system comprises a polar solvent.

11. The method of claim 1 wherein the Mn/Zr-based catalytic autoxidation system comprises a non-polar solvent.

12. The method of claim 1 wherein the method further comprises using oxygen as an oxidant.

13. The method of claim 1 wherein the oligomers are derived from pine lignin RCF oils.

14. The method of claim 1 wherein the oligomers are derived from poplar lignin RCF oils.

15. The method of any of claim 1, wherein the method is performed in a genetically engineered bacterium.

16. The method of claim 15 wherein the genetically engineered bacterium is Pseudomonas sp.

17. The method of claim 16 wherein the genetically engineered bacterium is ACB263.

18. The method of claim 12 wherein the method produces muconic acid.

19. The method of claim 12 wherein the method produces cis, cis-muconic acid.

20. The method of claim 17 wherein the genetically engineered bacterium is capable of metabolizing p-methoxylated aromatics.
